(19) United States
(12) Patent Application Publication
(10) Pub. No.: US 2024/0207283 A1
(43) Pub. Date: Jun. 27, 2024

(54) WATER SOLUBLE COMPLEX COMPOSITIONS AND METHODS THEREOF

(71) Applicant: VETERINARY PHARMACY CORPORATION, St. Peter, MN (US)

(72) Inventor: Patrick John SMITH, St. Peter, MN (US)

(21) Appl. No.: 18/565,487

(22) PCT Filed: May 27, 2022
(86) PCT No.: PCT/US2022/072617

§ 371 Date: Nov. 29, 2023

Related U.S. Application Data
(60) Provisional application No. 63/194,483, filed on May 28, 2021.

(51) Int. Cl. A61K 31/5415; A61K 9/10; A61K 47/12; A61K 47/18; A61K 47/22; A61K 47/34

(52) U.S. Cl. CPC A61K 31/5415; A61K 9/10; A61K 47/12; A61K 47/183; A61K 47/22; A61K 47/34

(57) ABSTRACT

The disclosure herein describes solubilizing an insoluble compound in water forming a complex with a hydrotrope and controlling the pH of the water or water-based solution. In some embodiments, methods for solubilizing a compound, such as meloxicam or piroxicam, in water are described. These methods can include forming a colloidal suspension of the compound, and then forming an aqueous colloidal suspension from the colloidal suspension.

### CROSS-REFERENCE TO RELATED APPLICATIONS

The present application claims priority to and benefit of U.S. Provisional Patent Application No. 63/194,483, filed May 28, 2021, and entitled “WATER SOLUBLE COMPLEX COMPOSITIONS AND METHODS THEREOF,” the entirety of which is herein incorporated by reference.

### FIELD

The present disclosure relates generally to formulating a water insoluble compound to be in a soluble form for treatment of animals.

### BACKGROUND

Meloxicam is a nonsteroidal anti-inflammatory drug (NSAID) which, in a mammal, can reduce hormones that cause inflammation and pain. It is generally known that meloxicam is insoluble in water, but slightly soluble in methanol. meloxicam has the molecular formula of C₁₄H₁₃N₃O₄S₂ and the following structure:

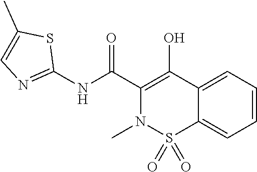 [SMILES: Cc1cnc(NC(=O)C2=C(O)c3ccccc3S(=O)(=O)N2C)s1]

### SUMMARY

Any or all portion(s) of any of the embodiments disclosed herein may be combined with any other portion(s) of any embodiment.

Some of the embodiments disclosed herein relate to methods for solubilizing a water insoluble compound in water or water-based colloidal suspension.

Some of the embodiments disclosed herein relate to methods for solubilizing meloxicam or piroxicam (oxicam derivative) in water or water-based colloidal suspension.

Some embodiments of a method for solubilizing meloxicam in water comprises obtaining the meloxicam; obtaining the water; obtaining a hydrotrope; and forming a colloidal suspension of meloxicam, wherein the forming of the colloidal suspension of meloxicam comprises mixing the meloxicam and the hydrotrope in the water; and forming an aqueous colloidal suspension of meloxicam from the colloidal suspension of meloxicam, wherein the forming of the aqueous colloidal suspension of meloxicam comprises obtaining a base; and adding an effective amount of the base to the colloidal suspension of meloxicam to change the pH of the colloidal suspension of meloxicam to be greater than 7.

In some embodiments of the method, the aqueous colloidal suspension of meloxicam includes: the meloxicam present at least in an amount of 7.15×10−6 g per mL of the aqueous colloidal suspension of meloxicam (or per mL of water).

In some embodiments of the method, the aqueous colloidal suspension of meloxicam includes: the hydrotrope being present in an amount of 0.001 g to 0.6 g per mL of the aqueous colloidal suspension of meloxicam (or per mL of water).

In some embodiments of the method, the aqueous colloidal suspension of meloxicam includes: the hydrotrope being present in an amount of 0.05 g to 0.6 g per mL of the aqueous colloidal suspension of meloxicam (or per mL of water).

In some embodiments of the method, the aqueous colloidal suspension of meloxicam includes: the hydrotrope being present in an amount of 0.001 g to 0.3 g per mL of the aqueous colloidal suspension of meloxicam (or per mL of water).

In some embodiments of the method, the aqueous colloidal suspension of meloxicam includes: the hydrotrope being present in an amount of 0.05 g to 0.3 g per mL of the aqueous colloidal suspension of meloxicam (or per mL of water).

In some embodiments, the hydrotrope being present can be 0.01 g to 0.6 g per mL of the aqueous colloidal suspension of meloxicam (or per mL of water).

In some embodiments, the hydrotrope being present can be 0.06 g to 0.6 g per mL of the aqueous colloidal suspension of meloxicam (or per mL of water).

In some embodiments, the hydrotrope being present can be 0.11 g to 0.6 g per mL of the aqueous colloidal suspension of meloxicam (or per mL of water).

In some embodiments, the hydrotrope being present can be 0.16 g to 0.6 g per mL of the aqueous colloidal suspension of meloxicam (or per mL of water).

In some embodiments, the hydrotrope being present can be 0.21 g to 0.6 g per mL of the aqueous colloidal suspension of meloxicam (or per mL of water).

In some embodiments, the hydrotrope being present can be 0.26 g to 0.6 g per mL of the aqueous colloidal suspension of meloxicam (or per mL of water).

In some embodiments, the hydrotrope being present can be 0.31 g to 0.6 g per mL of the aqueous colloidal suspension of meloxicam (or per mL of water).

In some embodiments, the hydrotrope being present can be 0.36 g to 0.6 g per mL of the aqueous colloidal suspension of meloxicam (or per mL of water).

In some embodiments, the hydrotrope being present can be 0.41 g to 0.6 g per mL of the aqueous colloidal suspension of meloxicam (or per mL of water).

In some embodiments, the hydrotrope being present can be 0.46 g to 0.6 g per mL of the aqueous colloidal suspension of meloxicam (or per mL of water).

In some embodiments, the hydrotrope being present can be 0.51 g to 0.6 g per mL of the aqueous colloidal suspension of meloxicam (or per mL of water).

In some embodiments, the hydrotrope being present can be 0.56 g to 0.6 g per mL of the aqueous colloidal suspension of meloxicam (or per mL of water).

In some embodiments, the hydrotrope being present can be 0.01 g to 0.55 g per mL of the aqueous colloidal suspension of meloxicam (or per mL of water).

In some embodiments, the hydrotrope being present can be 0.01 g to 0.5 g per mL of the aqueous colloidal suspension of meloxicam (or per mL of water).

In some embodiments, the hydrotrope being present can be 0.01 g to 0.45 g per mL of the aqueous colloidal suspension of meloxicam (or per mL of water).

In some embodiments, the hydrotrope being present can be 0.01 g to 0.4 g per mL of the aqueous colloidal suspension of meloxicam (or per mL of water).

In some embodiments, the hydrotrope being present can be 0.01 g to 0.35 g per mL of the aqueous colloidal suspension of meloxicam (or per mL of water).

In some embodiments, the hydrotrope being present can be 0.01 g to 0.3 g per mL of the aqueous colloidal suspension of meloxicam (or per mL of water).

In some embodiments, the hydrotrope being present can be 0.01 g to 0.25 g per mL of the aqueous colloidal suspension of meloxicam (or per mL of water).

In some embodiments, the hydrotrope being present can be 0.01 g to 0.2 g per mL of the aqueous colloidal suspension of meloxicam (or per mL of water).

In some embodiments, the hydrotrope being present can be 0.01 g to 0.15 g per mL of the aqueous colloidal suspension of meloxicam (or per mL of water).

In some embodiments, the hydrotrope being present can be 0.01 g to 0.1 g per mL of the aqueous colloidal suspension of meloxicam (or per mL of water).

In some embodiments, the hydrotrope being present can be 0.01 g to 0.05 g per mL of the aqueous colloidal suspension of meloxicam (or per mL of water).

In some embodiments of the method, the aqueous colloidal suspension of meloxicam, a concentration of the meloxicam is 0.000715% to 11.80%, wherein the meloxicam concentration is based on the formula, 100× (mass of meloxicam/volume of the aqueous colloidal suspension of meloxicam). In some embodiments of the method, the aqueous colloidal suspension of meloxicam has a meloxicam concentration of 0.000715% to 9.3%.

In some embodiments of the method, the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

In some embodiments of the method, the hydrotrope comprises an alkali metal salt. In some embodiments of the method, the alkali metal salt comprises a lithium salt. In some embodiments of the method, the alkali metal salt comprises a sodium salt. In some embodiments of the method, the alkali metal salt comprises a potassium salt. In some embodiments of the method, the alkali metal salt comprises a calcium salt.

In some embodiments of the method, the hydrotrope comprises at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N,N-dimethyl benzamide paraaminobenzoic acid hydrochloride, procaine hydrochloride, sodium alkanoate, urea, N,N-dimethylurea, resorcinol, pyrogallol, catechol, a,b-napthols, N-diethylnicotinamide (DENA) and N,N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

In some embodiments of the method, the base comprises a non-conventional Lewis base.

In some embodiments of the method, the base comprises a non-conventional hydroxide Lewis base.

In some embodiments of the method, the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

In some embodiments of the method, the forming of the aqueous colloidal suspension of meloxicam further comprises obtaining a pH buffer; and adding the pH buffer to the colloidal suspension of meloxicam.

In some embodiments of the method, the aqueous colloidal suspension of meloxicam includes: the pH buffer present in an amount of 0.001 g to 0.14 g per mL of the aqueous colloidal suspension of meloxicam.

In some embodiments of the method, the aqueous colloidal suspension of meloxicam includes the meloxicam present at least in an amount of 7.15×10−6 g per mL of the aqueous colloidal suspension of meloxicam, the hydrotrope being present in an amount of 0.001 g to 0.6 g per mL of the aqueous colloidal suspension of meloxicam; and the pH buffer present in an amount of 0.001 g to 0.14 g per mL of the aqueous colloidal suspension of meloxicam.

In some embodiments of the method, the pH buffer comprises at least one of glycine, arginine, or a combination thereof.

In some embodiments of the method, the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

In some embodiments of the method, the hydrotrope is sodium benzoate in an amount of 0.001 g to 0.6 g per mL of the aqueous colloidal suspension of meloxicam; and the pH buffer is glycine in an amount of at least 0.01 g per mL of the aqueous colloidal suspension of meloxicam.

In some embodiments of the method, the hydrotrope is sodium salicylate in an amount of at least 0.2 g per mL of the aqueous colloidal suspension of meloxicam; and the pH buffer is glycine in an amount of at least 0.01 g per mL of the aqueous colloidal suspension of meloxicam.

In some embodiments of the method, the hydrotrope is niacinamide in an amount of 0.001 g to 0.6 g per mL of the aqueous colloidal suspension of meloxicam; and the pH buffer is glycine in an amount of at least 0.01 g per mL of the aqueous colloidal suspension of meloxicam.

In some embodiments of the method, the hydrotrope is urea in an amount of 0.001 g to 0.6 g per mL of the aqueous colloidal suspension of meloxicam; and the pH buffer is glycine in an amount of at least 0.01 g per mL of the aqueous colloidal suspension of meloxicam.

In some embodiments of the method, the hydrotrope is phenylalanine in an amount of 0.001 g to 0.6 g per mL of the aqueous colloidal suspension of meloxicam; and the pH buffer is glycine in an amount of at least 0.01 g per mL of the aqueous colloidal suspension of meloxicam.

In some embodiments of the method, the obtaining the meloxicam comprises obtaining a tablet comprising the meloxicam as an active pharmaceutical ingredient (API).

In some embodiments, a method for treating illness in an animal comprises forming a medicated water-based colloidal suspension, wherein the forming the medicated water-based colloidal suspension comprises the method of solubilizing meloxicam in water; and providing the medicated water-based colloidal suspension to the animal.

In some embodiments of the method, the animal is at least one of a pig, poultry, a cattle, a sheep, a horse, a dog, or a cat.

In some embodiments of the method, the animal is a farm animal.

In some embodiments, an aqueous colloidal suspension of meloxicam comprises water; meloxicam, wherein the meloxicam is present at least in an amount of 7.15×10−6 g per mL of the water; a hydrotrope, wherein the hydrotrope is present in an amount of 0.001 g to 0.6 g per mL of the water; and a base, wherein the aqueous colloidal suspension of meloxicam has a pH of greater than 7.

In some embodiments of the aqueous colloidal suspension of meloxicam, a concentration of the meloxicam is 0.000715% to 11.80%. In some embodiments of the aqueous colloidal suspension of meloxicam, a concentration of the meloxicam is 0.000715% to 9.3%. In some embodiments, the aqueous colloidal suspension of meloxicam has a meloxicam concentration of 1.0%-12%. In some embodiments, the aqueous colloidal suspension of meloxicam has a meloxicam concentration of 2.0%-12%. In some embodiments, the aqueous colloidal suspension of meloxicam has a meloxicam concentration of 3.0%-12%. In some embodiments, the aqueous colloidal suspension of meloxicam has a meloxicam concentration of 4.0%-12%. In some embodiments, the aqueous colloidal suspension of meloxicam has a meloxicam concentration of 5.0%-12%. In some embodiments, the aqueous colloidal suspension of meloxicam has a meloxicam concentration of 6.0%-12%. In some embodiments, the aqueous colloidal suspension of meloxicam has a meloxicam concentration of 7.0%-12%. In some embodiments, the aqueous colloidal suspension of meloxicam has a meloxicam concentration of 8.0%-12%. In some embodiments, the aqueous colloidal suspension of meloxicam has a meloxicam concentration of 9.0%-12%. In some embodiments, the aqueous colloidal suspension of meloxicam has a meloxicam concentration of 10.0%-12%. In some embodiments, the aqueous colloidal suspension of meloxicam has a meloxicam concentration of 11.0%-12%. In some embodiments, the aqueous colloidal suspension of meloxicam has a meloxicam concentration of 1.0%-2.0%. In some embodiments, the aqueous colloidal suspension of meloxicam has a meloxicam concentration of 1.0%-3.0%. In some embodiments, the aqueous colloidal suspension of meloxicam has a meloxicam concentration of 1.0%-4.0%. In some embodiments, the aqueous colloidal suspension of meloxicam has a meloxicam concentration of 1.0%-5.0%. In some embodiments, the aqueous colloidal suspension of meloxicam has a meloxicam concentration of 1.0%-6.0%. In some embodiments, the aqueous colloidal suspension of meloxicam has a meloxicam concentration of 1.0%-7.0%. In some embodiments, the aqueous colloidal suspension of meloxicam has a meloxicam concentration of 1.0%-8.0%. In some embodiments, the aqueous colloidal suspension of meloxicam has a meloxicam concentration of 1.0%-9.0%. In some embodiments, the aqueous colloidal suspension of meloxicam has a meloxicam concentration of 1.0%-10%. In some embodiments, the aqueous colloidal suspension of meloxicam has a meloxicam concentration of 1.0%-11%. In some embodiments, the aqueous colloidal suspension of meloxicam has a meloxicam concentration of 1.0%-12%. In some embodiments, the aqueous colloidal suspension of meloxicam has a meloxicam concentration of 2.0%-3.0%. In some embodiments, the aqueous colloidal suspension of meloxicam has a meloxicam concentration of 2.0%-4.0%. In some embodiments, the aqueous colloidal suspension of meloxicam has a meloxicam concentration of 2.0%-5.0%. In some embodiments, the aqueous colloidal suspension of meloxicam has a meloxicam concentration of 2.0%-6.0%. In some embodiments, the aqueous colloidal suspension of meloxicam has a meloxicam concentration of 2.0%-7.0%. In some embodiments, the aqueous colloidal suspension of meloxicam has a meloxicam concentration of 2.0%-8.0%. In some embodiments, the aqueous colloidal suspension of meloxicam has a meloxicam concentration of 2.0%-9.0%. In some embodiments, the aqueous colloidal suspension of meloxicam has a meloxicam concentration of 2.0%-10%. In some embodiments, the aqueous colloidal suspension of meloxicam has a meloxicam concentration of 2.0%-11%. In some embodiments, the aqueous colloidal suspension of meloxicam has a meloxicam concentration of 2.0%-12%. In some embodiments, the aqueous colloidal suspension of meloxicam has a meloxicam concentration of 3.0%-4.0%. In some embodiments, the aqueous colloidal suspension of meloxicam has a meloxicam concentration of 3.0%-5.0%. In some embodiments, the aqueous colloidal suspension of meloxicam has a meloxicam concentration of 3.0%-6.0%. In some embodiments, the aqueous colloidal suspension of meloxicam has a meloxicam concentration of 3.0%-7.0%. In some embodiments, the aqueous colloidal suspension of meloxicam has a meloxicam concentration of 3.0%-8.0%. In some embodiments, the aqueous colloidal suspension of meloxicam has a meloxicam concentration of 3.0%-9.0%. In some embodiments, the aqueous colloidal suspension of meloxicam has a meloxicam concentration of 3.0%-10%. In some embodiments, the aqueous colloidal suspension of meloxicam has a meloxicam concentration of 3.0%-11%. In some embodiments, the aqueous colloidal suspension of meloxicam has a meloxicam concentration of 3.0%-12%. In some embodiments, the aqueous colloidal suspension of meloxicam has a meloxicam concentration of 4.0%-5.0%. In some embodiments, the aqueous colloidal suspension of meloxicam has a meloxicam concentration of 4.0%-6.0%. In some embodiments, the aqueous colloidal suspension of meloxicam has a meloxicam concentration of 4.0%-7.0%. In some embodiments, the aqueous colloidal suspension of meloxicam has a meloxicam concentration of 4.0%-8.0%. In some embodiments, the aqueous colloidal suspension of meloxicam has a meloxicam concentration of 4.0%-9.0%. In some embodiments, the aqueous colloidal suspension of meloxicam has a meloxicam concentration of 4.0%-10%. In some embodiments, the aqueous colloidal suspension of meloxicam has a meloxicam concentration of 4.0%-11%. In some embodiments, the aqueous colloidal suspension of meloxicam has a meloxicam concentration of 4.0%-12%. In some embodiments, the aqueous colloidal suspension of meloxicam has a meloxicam concentration of 5.0%-6.0%. In some embodiments, the aqueous colloidal suspension of meloxicam has a meloxicam concentration of 5.0%-7.0%. In some embodiments, the aqueous colloidal suspension of meloxicam has a meloxicam concentration of 5.0%-8.0%. In some embodiments, the aqueous colloidal suspension of meloxicam has a meloxicam concentration of 5.0%-9.0%. In some embodiments, the aqueous colloidal suspension of meloxicam has a meloxicam concentration of 5.0%-10%. In some embodiments, the aqueous colloidal suspension of meloxicam has a meloxicam concentration of 5.0%-11%. In some embodiments, the aqueous colloidal suspension of meloxicam has a meloxicam concentration of 5.0%-12%. In some embodiments, the aqueous colloidal suspension of meloxicam has a meloxicam concentration of 6.0%-7.0%. In some embodiments, the aqueous colloidal suspension of meloxicam has a meloxicam concentration of 6.0%-8.0%. In some embodiments, the aqueous colloidal suspension of meloxicam has a meloxicam concentration of 6.0%-9.0%. In some embodiments, the aqueous colloidal suspension of meloxicam has a meloxicam concentration of 6.0%-10%. In some embodiments, the aqueous colloidal suspension of meloxicam has a meloxicam concentration of 6.0%-11%. In some embodiments, the aqueous colloidal suspension of meloxicam has a meloxicam concentration of 6.0%-12%. In some embodiments, the aqueous colloidal suspension of meloxicam has a meloxicam concentration of 7.0%-8.0%. In some embodiments, the aqueous colloidal suspension of meloxicam has a meloxicam concentration of 7.0%-9.0%. In some embodiments, the aqueous colloidal suspension of meloxicam has a meloxicam concentration of 7.0%-10%. In some embodiments, the aqueous colloidal suspension of meloxicam has a meloxicam concentration of 7.0%-11%. In some embodiments, the aqueous colloidal suspension of meloxicam has a meloxicam concentration of 7.0%-12%. In some embodiments, the aqueous colloidal suspension of meloxicam has a meloxicam concentration of 8.0%-9.0%. In some embodiments, the aqueous colloidal suspension of meloxicam has a meloxicam concentration of 8.0%-10%. In some embodiments, the aqueous colloidal suspension of meloxicam has a meloxicam concentration of 8.0%-11%. In some embodiments, the aqueous colloidal suspension of meloxicam has a meloxicam concentration of 8.0%-12%. In some embodiments, the aqueous colloidal suspension of meloxicam has a meloxicam concentration of 9.0%-10%. In some embodiments, the aqueous colloidal suspension of meloxicam has a meloxicam concentration of 9.0%-11%. In some embodiments, the aqueous colloidal suspension of meloxicam has a meloxicam concentration of 9.0%-12%. In some embodiments, the aqueous colloidal suspension of meloxicam has a meloxicam concentration of 10%-11%. In some embodiments, the aqueous colloidal suspension of meloxicam has a meloxicam concentration of 10%-12%. In some embodiments, the aqueous colloidal suspension of meloxicam has a meloxicam concentration of 11%-12%.

In some embodiments of the aqueous colloidal suspension of meloxicam, the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

In some embodiments of the aqueous colloidal suspension of meloxicam, the base comprises sodium hydroxide.

In some embodiments of the aqueous colloidal suspension of meloxicam of claim 34, further comprising a pH buffer, wherein the pH buffer is present in an amount of 0.001 g to 0.14 g per mL of the water.

In some embodiments of the aqueous colloidal suspension of meloxicam, the pH buffer comprises at least one of glycine, arginine, or a combination thereof.

In some embodiments of the aqueous colloidal suspension of meloxicam, the hydrotrope is sodium salicylate in an amount of at least 0.2 g per mL of the aqueous colloidal suspension of meloxicam; and the pH buffer is glycine in an amount of at least 0.01 g per mL of the aqueous colloidal suspension of meloxicam.

In some embodiments, a method for solubilizing meloxicam in a stock colloidal suspension comprises obtaining the meloxicam; obtaining the stock colloidal suspension; obtaining a hydrotrope; forming a colloidal suspension of meloxicam, wherein the forming of the colloidal suspension of meloxicam comprises mixing the meloxicam and the hydrotrope in the stock colloidal suspension; and forming a medicated stock colloidal suspension from the colloidal suspension of meloxicam, wherein the forming of the medicated stock colloidal suspension comprises obtaining a base; and adding an effective amount of the base to the colloidal suspension of meloxicam to change the pH of the colloidal suspension of meloxicam to at least 8.

In some embodiments, the method further comprising mixing the medicated stock colloidal suspension with water.

In some embodiments, a method for treating illness in an animal comprises forming a medicated water-based colloidal suspension, wherein the forming the medicated water-based colloidal suspension comprises the method of solubilizing meloxicam in a stock colloidal suspension; and providing the medicated water-based colloidal suspension to the animal.

In some embodiments of the method, the animal is at least one of a pig, poultry, a cattle, a sheep, a horse, a dog, or a cat.

In some embodiments, a method for solubilizing piroxicam in water comprises obtaining the piroxicam; obtaining the water; obtaining a hydrotrope; and forming a colloidal suspension of piroxicam, wherein the forming of the colloidal suspension of piroxicam comprises mixing the piroxicam and the hydrotrope in the water; and forming an aqueous colloidal suspension of piroxicam from the colloidal suspension of meloxicam, wherein the forming of the aqueous colloidal suspension of piroxicam comprises obtaining a base; and adding an effective amount of the base to the colloidal suspension of piroxicam to change the pH of the colloidal suspension of piroxicam to be greater than 7.

In some embodiments, the medicated stock colloidal suspension is mixed with water at a mixing ratio of 1:50 (the medicated stock colloidal suspension:water). In some embodiments, the medicated stock colloidal suspension is mixed with water at a mixing ratio of 1:64 (the medicated stock colloidal suspension:water). In some embodiments, the medicated stock colloidal suspension is mixed with water at a mixing ratio of 1:100 (the medicated stock colloidal suspension:water). In some embodiments, the medicated stock colloidal suspension is mixed with water at a mixing ratio of 1:128 (the medicated stock colloidal suspension:water). In some embodiments, the medicated stock colloidal suspension is mixed with water at a mixing ratio of 1:200 (the medicated stock colloidal suspension:water). In some embodiments, the medicated stock colloidal suspension is mixed with water at a mixing ratio of 1:500 (the medicated stock colloidal suspension:water).

### BRIEF DESCRIPTION OF THE DRAWINGS

References are made to the accompanying drawings that form a part of this disclosure and that illustrate embodiments in which the systems and methods described in this Specification can be practiced.

FIG. 1 is a schematic diagram and flowchart showing an embodiment for providing a medicated water solution.

### DETAILED DESCRIPTION

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

The embodiments disclosed herein are directed towards formulating a water insoluble compound (e.g., meloxicam) to be in a soluble form, via forming a complex composition with a hydrotrope and affecting the pH of the mixture to form a colloidal suspension. The resultant colloidal suspension can be further mixed with water or water-based solution (e.g., stock) for providing the resultant medicated water or medicated water-based solution to treat animals.

Colloidal suspension, as used herein, means a mixture or a complex composition in which a first substance is divided into minute particles (called colloidal particles) and dispersed throughout a second substance. The first and second substances are present as larger particles than those found in a traditional solution, but these larger particles are too small to be seen with a microscope. When the colloidal suspension is in water, it is called an aqueous colloidal suspension. There are generally no strict boundaries on the size of colloidal particles, but they can be from 10-9 m to 10-6 m in size. A colloidal suspension is not a true solution but it is also not a “true suspension” either because the colloidal particles do not settle out like a true suspension will over time. The colloidal suspension will also display a Tyndal effect phenomenon and Brownian motion. As used herein, the term “solution” is used to describe a colloidal suspension (and for clarity, the term “true solution” is used for a type of mixture which does not include a colloidal suspension). The colloidal suspension can be formed via use of hydrotropes.

Hydrotropes (also called “hydrotropic agent,” e.g., sodium benzoate) have a property to increase the solubility of insoluble molecules. A common feature of many hydrotropes is a small aromatic ring that allows it to have this effect. The interaction may not be entirely colloidal. Instead, the hydrotropic agent has a weak interaction with a solute molecule (which may be generally insoluble) that allow a complex to form. This complex is what becomes soluble. The efficiency of the hydrotropic agent depends on a balance between its hydrophilic and hydrophobic pieces. The larger the hydrophobic part of the agent the better its efficiency. Another common feature of hydrotropic agents is their own high solubility (e.g., in water). Other mechanisms that can be important to better efficiency is self-aggregation potential, structure breaker, and structure maker, and ability to form a micelle like structure. There can also be an importance of a minimum hydrotropic agent concentration needed to achieve solubility of a solute. This minimum concentration is a concentration at which the hydrotrope aggregates. At higher concentrations, the hydrotropes begin to decrease the cloud point of the solution.

In an example, 500 mg of meloxicam was obtained and mixed with 100 ml of water, with an addition of 10 g of sodium benzoate NF. The mixture was agitated, forming a suspension. A further 5 g of sodium benzoate was added with no change. The initial pH of the mixture was noted to be at ˜5.0. Upon adding 0.08 g sodium hydroxide to raise the pH to 8.0, a clearing solution began appearing. Further NaOH was added until the pH reached 10.0, and a clear dark yellow single phased solution was achieved. The stability of this clear solution was tested (to see whether it would be brought out of solution) by adding water from various sources (e.g., tap water, distilled water, etc.). Surprisingly, the addition or disturbance using more water did not cause any precipitation of the product. 1M HCL acid was added in small amounts to see how decreases in pH would affect the product. The product remained mostly in solution until the pH dropped below 8.0 and a suspension began reforming. This experiment was repeated to ensure reproducibility. Similar results were seen.

In another example, a bulk meloxicam powder was obtained and the limiting returns of solubilization was found to be based on the amount of sodium benzoate required to achieve a solution. A ratio of ˜30 g sodium benzoate:1 g of meloxicam base powder in 100 ml solution was able to achieve optimal solution clarity with addition of sodium hydroxide. The main limiting factor in this example was determined to be the amount of sodium benzoate required eventually exceeding its water solubility (˜62.9 g/100 ml) as the meloxicam concentration was increased. In this example, a maximum concentration of 2 g/100 ml of meloxicam was achieved using sodium benzoate as the sole hydrotrope.

An exemplary method was confirmed as being viable for concentrations up to 15-20 mg/ml of meloxicam using meloxicam base powder. In various examples, other hydrotropes were used to replicate the formation of a clear solution, and to determine whether other hydrotrope or combinations may have similar or stronger effects.

In an example, it was determined that sodium citrate, both with and without sodium benzoate, even in concentrations up to 50% did not create a solution. It did form a suspension but eventually upon further addition of salt it saturated the solution and precipitated out.

In another example, sodium salicylate was used as a hydrotrope. This was substantially more successful (besides sodium benzoate) in forming a clear solution. This was found to solubilize meloxicam at a ratio by itself of ˜15-20 g sodium salicylate:1 g meloxicam in 100 ml of solution.

In other examples, salicylic acid was used for converting to sodium salicylate using NaOH. In other examples, sodium salicylate powder was used (which achieved similar results but without the requirements of the sodium hydroxide (NaOH)).

In other examples, acetylsalicylic acid was used as the hydrotrope. In these examples, a clear solution was achieved. It is noted that an increased amount of NaOH was needed to achieve the rise in pH when acetylsalicylic acid was the hydrotrope. It is also noted that acetylsalicylic acid tended to form acetic acid byproducts as it was cleaved.

The examples where sodium salicylate was used as the hydrotrope yielded some of the highest concentration of solution achieving a 3.5% meloxicam solution using ˜45 g of sodium salicylate by itself along with NaOH to achieve a pH above 9.0.

In another example, sodium salicylate and sodium benzoate were used together to see whether there is a synergistic enhancement or improvement. It was found that a successful 1% meloxicam solution can be made using 15 g sodium benzoate/100 ml and 10 g sodium salicylate. This confirmed that combining hydrotropes can reduce the requirement of sodium benzoate concentration. To see whether this synergy would allow a higher than 2% solution concentration. It was found that by using ˜25 g of sodium salicylate and ˜30 g of sodium benzoate, a 3% meloxicam solution was obtainable.

In another example, meloxicam in a 1% and 2% solution (colloidal suspension) was achieved upon adding a ratio of roughly ˜20 g of urea per 1 g of meloxicam. Therefore, urea can serve as a hydrotrope in some embodiments.

In some examples, niacinamide is or is one of the hydrotrope(s) for solubilizing meloxicam in 1%, 2%, 3%, and 4.6% solutions (colloidal suspensions). The established ratio of this hydrotrope to meloxicam was ˜5 g of niacinamide: 1 g of meloxicam in 100 ml of solution, wherein the clear solution begins to form once the pH is increased to above 8.0. In some examples, the clear solution begins to form once the pH is increased to above 9.0. In some examples, the clear solution begins to form once the pH is in a range from 8.0-9.0.

Additional examples were successful in solubilizing meloxicam in a 9% solution. Niacinamide was found to successfully solubilize meloxicam and achieve surprisingly high solubilized concentration of at least 9% (e.g., 9% to 9.24%). The established ratio of this hydrotrope to meloxicam was ˜5 g of niacinamide: 1 g of meloxicam in 100 ml of solution, wherein the clear solution begins to form once the pH is increased to above 9.0. Of note, in this example (and also as needed in other examples) heating was required at 35 degrees Celsius to warm the solution and fully solubilize the product. It might be unstable as the 9.24% product began precipitating out shortly after it was solubilized once the solution began to cool to room temperature. Accordingly, maintaining the temperature above room temperature (or in some embodiments, at a temperature of 35 degrees Celsius or higher) can be required in some embodiments to maintain the solubility property of the meloxicam complex. The 9.0% was slightly more stable as it cooled, however it can begin to form precipitates.

Additional examples were successful in solubilizing meloxicam in a 11.8% solution. Niacinamide was found to successfully solubilize meloxicam and achieve surprisingly high solubilized concentration of at least 11% and some examples have reached higher concentrations, such as 12% (e.g., in some examples, achievable concentrations are from 1.0% to 12%). In one specific example, the concentration of 117.98 mg/ml (11.8% (wt/vol)) was reached. The established ratio of this hydrotrope to meloxicam was ˜5 g of niacinamide: 1 g of meloxicam in 100 ml of solution, wherein the clear solution begins to form once the pH is increased to above 8.5 (e.g., from 8.5 to 9.0). Arginine was used as the base and buffer system in some examples. In some examples, NaOH was not used as the base and buffer system. Of note, in some examples, heating was required at 35 degrees Celsius to warm the solution and fully solubilize the product.

In some examples, niacinamide was used to solubilize the piroxicam, which is an NSAID in the same structural class as meloxicam) to form an 8% solution. It was found to successfully solubilize the piroxicam and achieve a solubilized concentration of ˜8%. The established ratio of this hydrotrope was similar to meloxicam at the ˜5 g of niacinamide: 1 g of Piroxicam in 100 ml of solution, wherein the clear solution begins to form once the pH is increased to above 9.0. It is likely that because of the similar characteristics the piroxicam shares with meloxicam that it would also be amenable to solubilization with the other hydrotropes as well.

In other examples, other hydrotropes have been tested and was determined to also provide successful increased solubilities of meloxicam and piroxicam. Amino acids such as Tryptophan, Proline, Tyrosine, and Phenylalanine were chosen due to their structure and potential to be hydrotropic agents. As well, Niacin and Sodium Acetate were also experimented with to solubilize meloxicam solutions. Phenylalanine, Proline, and Niacin were the only agents found to successfully solubilize meloxicam in a significant amount greater than 5 mg/ml. Sodium acetate and Tyrosine nearly achieved solutions but were unable to achieve the desired homogenous clear liquid state.

In another example, meloxicam finished dosage form tablets were used to extract meloxicam from the tablets into a solution (colloidal suspension) after allowing the insoluble debris from the meloxicam tablets to settle out. Upon using this solubilization process a three phased solution was created with the colloidal suspension of interest in the center. The product achieved a roughly 90% concentration of the theoretical concentration.

In additional examples, the stability of meloxicam yielded were studied (due to the method having an unbuffered system). These examples studied whether there was hydrolysis of the meloxicam molecule that could lead to instability of the meloxicam in the colloidal suspension. Such hydrolysis might be seen due to photosensitivity or acidic environments. In an example, a 0.5-2% buffer of glycine was added and it was determined that the glycine buffer did not impact the solubilization of the product. In another example, 1% arginine was used to act as both a buffer and to achieve a target pH (pH>7) to replace sodium hydroxide. In these examples, at least with regards to the solubilization of meloxicam from tablets, the three-phase layer was found to be more easily separated upon the addition of simethicone defoamer.

The following are some example formulas for the meloxicam complex water-soluble products:
1) meloxicam Solution from USP powder
   a. meloxicam 1 g/100 ml
   b. sodium benzoate 30 g/100 ml
   c. glycine 1 g/100 ml
   d. NaOH q.s. pH target 9-11
   e. H₂O q.s.
2) meloxicam Solution from USP powder
   a. meloxicam 1 g/100 ml
   b. sodium salicylate or acetylsalicylic acid 20 g/100 ml
   C. glycine 1 g/100 ml
   d. NaOH q.s. pH target 9-11
   e. H₂O q.s.
3) meloxicam Solution from USP powder
   a. meloxicam 1 g/100 ml
   b. sodium salicylate 10 g/100 ml
   c. sodium benzoate 15 g/100 ml
   d. glycine 1 g/100 ml
   e. NaOH q.s. pH target 9-11
   f. H₂O q.s.
4) meloxicam Solution from USP Powder
   a. meloxicam 1 g/100 ml
   b. hydrotrope 20-30 g/100 ml
   C. arginine 0.8-1 g/100 ml
   d. H₂O q.s.
   e. pH target 9-11
5) meloxicam solution from Tablet formulation
   a. meloxicam tablets 15 mg 66.7 tablets/100 ml
   b. sodium benzoate 31 g/100 ml
   C. simethicone defoamer 0.02 ml/100 ml
   d. NaOH 0.55 g/100 ml
   e. glycine 2 g/100 ml
   f. H₂O q.s.
6) meloxicam solution USP powder
   a. meloxicam 1 g/100 ml
   b. niacinamide 5 g/100 ml
   C. NaOH q.s. pH target 9-11
   d. glycine 1 g/100 ml
   e. H₂O q.s.

Target Market Example: Livestock/animal(s) (e.g., pig, poultry, cattle, sheep, horse, dog, cat, etc.).

There are estimated to be about 6.41 million sows in the U.S. Market. Cross-sectional studies show that the within-herd prevalence of sow lameness is quite high and may range from 8.8% to 16.9%. This equates to a potential market of 564,000 to 1,083,000 animals requiring treatment at some point.

There are roughly 75 million hogs in the United States. Studies have shown anywhere from the percentage seen in sows to up to 40% or more of growing herd can experience lameness. This would equate to up to 30 million hogs potentially experiencing this debilitating disease at some point and requiring treatment.

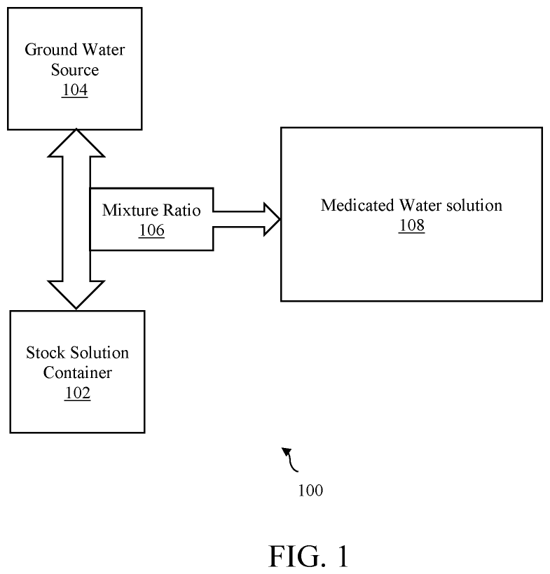

FIG. 1 shows a schematic diagram showing an embodiment of a treatment administration system and process. According to this system and process, meloxicam or piroxicam is provided for animal(s) in a diluted form, delivered via a water medication system 100. A container with concentrated meloxicam solution or dry powder packet blend is added to stock solution container 102. Medication is diluted in X gallons of Stock Solution. This product is mixed with a ground water source 104 at a dilution rate or ratio 106 (e.g., 1 gallon Stock Solution:128 gallons Water). The resulting Medicated Water solution 108 is provided to the animal(s). In some embodiments, the Stock Solution to Water mixing ratio is 1:50, 1:64, 1:100, 1:128, 1:200, or 1:500.

### pH Stability Tests

Surprisingly, various examples have been found to be very stable over various ranges of pH and also over a long time. For example, the following exemplary pH stress test was performed.

Samples of the Meloxicam Colloidal Solution according to some embodiments were stress tested using 1M HCL acid to assess at what pH the colloid was broken. In at least some examples, the Meloxicam colloidal suspension is stable below a pH of 7 when acidic media is added, but below a pH of 4 the colloid breaks and a suspension is formed that begins settling out. From an initial pH of the solution being 8.3, HCL acid was added dropwise to a final pH of 3.8. The colloidal suspension showed no significant change until the pH was measured at 5.6 at which point it began to turn from a clear solution to a slightly cloudy solution. No settling of particulates to the bottom was noticed (particulates settling to the bottom would be indicative of a suspension being broken/formed). From pH of 5.6, pH was further lowered to pH of 4.5. At this point, settling of product began to occur. Accordingly, pH of 4.5 was determined to be the breaking point of the colloid system. Further, the solution turned steadily cloudier as the pH was lowered even further. At the final pH of 3.8, settling of product became very noticeable.

Samples of the Meloxicam Colloidal Solution were taken at various pH points of 4.8, 4.2, and 3.8. These samples were diluted with water to assess settling out of the system in a simulated stock solution environment. Settling of product occurred in these samples below various “expected” colloidal pH breaking points. In some samples, pH of 4.8 did not have any easily noticeable settling of product even with the cloudiness of the solution due to the lowered pH.

### Stability Tests

Some samples were kept for a long duration of time to determine the stability of the colloidal suspension over time (up to around 17 months and 2 weeks) after formation. In these samples, there were no color changes, sedimentations, or other visual defects to the colloidal suspensions. These were very surprising and good results as they showed that the colloidal suspensions are stable over months. According to some embodiments, the stability can be adjusted such that the various length of time shown below can be combined to form ranges of time of stability of the colloidal suspensions. In some embodiments, the colloidal suspensions are stable for at least 17 months after formation. In some embodiments, the colloidal suspensions are stable for at least 16 months after formation. In some embodiments, the colloidal suspensions are stable for at least 15 months after formation. In some embodiments, the colloidal suspensions are stable for at least 14 months after formation. In some embodiments, the colloidal suspensions are stable for at least 13 months after formation. In some embodiments, the colloidal suspensions are stable for at least 12 months after formation. Further, it is expected that some embodiments of the colloidal suspensions can be stable for up to 13 months or can be stable for over 13 months and perhaps even longer. It has been found that some examples was stable for at least 17 months and 2 weeks since formulation. In some embodiments, the colloidal suspensions are stable for at least 11 months. In some embodiments, the colloidal suspensions are stable for at least 10 months. In some embodiments, the colloidal suspensions are stable for at least 9 months. In some embodiments, the colloidal suspensions are stable for at least 8 months. In some embodiments, the colloidal suspensions are stable for at least 7 months. In some embodiments, the colloidal suspensions are stable for at least 6 months. In some embodiments, the colloidal suspensions are stable for at least 5 months. In some embodiments, the colloidal suspensions are stable for at least 4 months. In some embodiments, the colloidal suspensions are stable for at least 3 months. In some embodiments, the colloidal suspensions are stable for at least 2 months. In some embodiments, the colloidal suspensions are stable for at least 1 month. In some embodiments, the colloidal suspensions are stable for up to 12 months after formation. In some embodiments, the colloidal suspensions are stable for up to 11 months after formation. In some embodiments, the colloidal suspensions are stable for up to 10 months after formation. In some embodiments, the colloidal suspensions are stable for up to 9 months after formation. In some embodiments, the colloidal suspensions are stable for up to 8 months after formation. In some embodiments, the colloidal suspensions are stable for up to 7 months after formation. In some embodiments, the colloidal suspensions are stable for up to 6 months after formation. In some embodiments, the colloidal suspensions are stable for up to 5 months after formation. In some embodiments, the colloidal suspensions are stable for up to 4 months after formation. In some embodiments, the colloidal suspensions are stable for up to 3 months after formation. In some embodiments, the colloidal suspensions are stable for up to 2 months after formation. In some embodiments, the colloidal suspensions are stable for up to 1 month after formation.

### ASPECTS

Various Aspects are described below. It is to be understood that any one or more of the features recited in the following Aspect(s) can be combined with any one or more other Aspect(s).

Aspect 1. A method for solubilizing meloxicam in water, comprising:
obtaining the meloxicam;
obtaining the water;
obtaining a hydrotrope; and
forming a colloidal suspension of meloxicam,
   wherein the forming of the colloidal suspension of meloxicam comprises:
      mixing the meloxicam and the hydrotrope in the water; and
      forming an aqueous colloidal suspension of meloxicam from the colloidal suspension of meloxicam,
         wherein the forming of the aqueous colloidal suspension of meloxicam comprises:
         obtaining a base; and
         adding an effective amount of the base to the colloidal suspension of meloxicam to change the pH of the colloidal suspension of meloxicam to be greater than 7.

Aspect 2. The method of Aspect 1, wherein the aqueous colloidal suspension of meloxicam includes:
the meloxicam present at least in an amount of 7.15×10⁻⁶ g per mL of the aqueous colloidal suspension of meloxicam.

Aspect 3. The method according to any of Aspects 1-2, wherein the aqueous colloidal suspension of meloxicam includes:
the hydrotrope being present in an amount of 0.001 g to 0.6 g per mL of the aqueous colloidal suspension of meloxicam.

Aspect 4. The method according to any of the preceding Aspects, wherein the aqueous colloidal suspension of meloxicam includes:
the hydrotrope being present in an amount of 0.05 g to 0.6 g per mL of the aqueous colloidal suspension of meloxicam.

Aspect 5. The method according to any of the preceding Aspects, wherein the aqueous colloidal suspension of meloxicam includes:
the hydrotrope being present in an amount of 0.001 g to 0.3 g per mL of the aqueous colloidal suspension of meloxicam.

Aspect 6. The method according to any of the preceding Aspects, wherein the aqueous colloidal suspension of meloxicam includes:
the hydrotrope being present in an amount of 0.05 g to 0.3 g per mL of the aqueous colloidal suspension of meloxicam.

Aspect 7. The method according to any of the preceding Aspects, wherein the aqueous colloidal suspension of meloxicam has a meloxicam concentration of 0.000715% to 12%, wherein the meloxicam concentration is based on the formula, 100× (mass of meloxicam/volume of the aqueous colloidal suspension of meloxicam).

Aspect 8. The method according to any of the preceding Aspects, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

Aspect 9. The method according to any of the preceding Aspects, wherein the hydrotrope comprises an alkali metal salt.

Aspect 10. The method of Aspect 9, wherein the alkali metal salt comprises a lithium salt.

Aspect 11. The method according to any of Aspects 9-10, wherein the alkali metal salt comprises a sodium salt.

Aspect 12. The method according to any of Aspects 9-11, wherein the alkali metal salt comprises a potassium salt.

Aspect 13. The method according to any of Aspects 9-12, wherein the alkali metal salt comprises a calcium salt.

Aspect 14. The method according to any of the preceding Aspects, wherein the hydrotrope comprises at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N, N-dimethyl benzamide, paraaminobenzoic acid hydrochloride, procaine hydrochloride, sodium alkanoate, urea and N, N-dimethylurea Resorcinol, pyrogallol, catechol, a,b-napthols, N-diethylnicotinamide (DENA), N, N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

Aspect 15. The method according to any of the preceding Aspects, wherein the base comprises a non-conventional Lewis base.

Aspect 16. The method according to any of the preceding Aspects, wherein the base comprises a non-conventional hydroxide Lewis base.

Aspect 17. The method according to any of the preceding Aspects, wherein the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

Aspect 18. The method according to any of the preceding Aspects, wherein the forming of the aqueous colloidal suspension of meloxicam further comprises:
obtaining a pH buffer; and
adding the pH buffer to the colloidal suspension of meloxicam.

Aspect 19. The method of Aspect 18, wherein the aqueous colloidal suspension of meloxicam includes:

the pH buffer present in an amount of 0.001 g to 0.14 g per mL of the aqueous colloidal suspension of meloxicam.

Aspect 20. The method of Aspect 19, wherein the aqueous colloidal suspension of meloxicam includes:
the meloxicam present at least in an amount of 7.15×10⁻⁶ g per mL of the aqueous colloidal suspension of meloxicam,
the hydrotrope being present in an amount of 0.001 g to 0.6 g per mL of the aqueous colloidal suspension of meloxicam; and
the pH buffer present in an amount of 0.001 g to 0.14 g per mL of the aqueous colloidal suspension of meloxicam.

Aspect 21. The method according to any of Aspects 19-20, wherein the pH buffer comprises at least one of glycine, arginine, or a combination thereof.

Aspect 22. The method according to any of Aspects 19-21, wherein the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

Aspect 23. The method according to any of Aspects 19-22, wherein:
the hydrotrope is sodium benzoate in an amount of 0.001 g to 0.6 g per mL of the aqueous colloidal suspension of meloxicam; and
the pH buffer is glycine in an amount of at least 0.01 g per mL of the aqueous colloidal suspension of meloxicam.

Aspect 24. The method according to any of Aspects 19-23, wherein:
the hydrotrope is sodium salicylate in an amount of at least 0.2 g per mL of the aqueous colloidal suspension of meloxicam; and
the pH buffer is glycine in an amount of at least 0.01 g per mL of the aqueous colloidal suspension of meloxicam.

Aspect 25. The method according to any of Aspects 19-24, wherein:
the hydrotrope is niacinamide in an amount of 0.001 g to 0.6 g per mL of the aqueous colloidal suspension of meloxicam; and
the pH buffer is glycine in an amount of at least 0.01 g per mL of the aqueous colloidal suspension of meloxicam.

Aspect 26. The method according to any of Aspects 19-25, wherein:
the hydrotrope is urea in an amount of 0.001 g to 0.6 g per mL of the aqueous colloidal suspension of meloxicam; and
the pH buffer is glycine in an amount of at least 0.01 g per mL of the aqueous colloidal suspension of meloxicam.

Aspect 27. The method according to any of Aspects 19-26, wherein:
the hydrotrope is phenylalanine in an amount of 0.001 g to 0.6 g per mL of the aqueous colloidal suspension of meloxicam; and
the pH buffer is glycine in an amount of at least 0.01 g per mL of the aqueous colloidal suspension of meloxicam.

Aspect 28. The method according to any of the preceding Aspects, wherein the obtaining the meloxicam comprises obtaining a tablet comprising the meloxicam as an active pharmaceutical ingredient (API).

Aspect 29. A method for treating illness in an animal, comprising:
forming a medicated water-based colloidal suspension,
   wherein the forming the medicated water-based colloidal suspension comprises:
      the method of solubilizing meloxicam in water according to any of Aspects 1-28; and
providing the medicated water-based colloidal suspension to the animal.

Aspect 30. The method of Aspect 29, wherein the animal is at least one of a pig, poultry, a cattle, a sheep, a horse, a dog, or a cat.

Aspect 31. The method of Aspect 29, wherein the animal is a farm animal.

Aspect 32. An aqueous colloidal suspension of meloxicam, comprising: water;
meloxicam,
   wherein the meloxicam is present at least in an amount of 7.15×10⁻⁶ g per mL of the water;
a hydrotrope,
   wherein the hydrotrope is present in an amount of 0.001 g to 0.6 g per mL of the water; and
a base,
wherein the aqueous colloidal suspension of meloxicam has a pH of greater than 7.

Aspect 33. The aqueous colloidal suspension of meloxicam of Aspect 32, comprising a concentration of the meloxicam of 0.000715% to 12%.

Aspect 34. The aqueous colloidal suspension of meloxicam according to any of Aspects 32-33, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

Aspect 35. The aqueous colloidal suspension of meloxicam according to any of Aspects 32-34, wherein the base comprises sodium hydroxide.

Aspect 36. The aqueous colloidal suspension of meloxicam according to any of Aspects 32-35, further comprising:
a pH buffer,
   wherein the pH buffer is present in an amount of 0.001 g to 0.14 g per mL of the water.

Aspect 37. The aqueous colloidal suspension of meloxicam of Aspect 36, wherein the pH buffer comprises at least one of glycine, arginine, or a combination thereof.

Aspect 38. The aqueous colloidal suspension of meloxicam according to any of Aspects 36-37, wherein:
the hydrotrope is sodium salicylate in an amount of at least 0.2 g per mL of the aqueous colloidal suspension of meloxicam; and
the pH buffer is glycine in an amount of at least 0.01 g per mL of the aqueous colloidal suspension of meloxicam.

Aspect 39. A method for solubilizing meloxicam in a stock colloidal suspension, comprising:
obtaining the meloxicam;
obtaining the stock colloidal suspension;
obtaining a hydrotrope;
forming a colloidal suspension of meloxicam,
   wherein the forming of the colloidal suspension of meloxicam comprises:
      mixing the meloxicam and the hydrotrope in the stock colloidal suspension; and
forming a medicated stock colloidal suspension from the colloidal suspension of meloxicam,
   wherein the forming of the medicated stock colloidal suspension comprises:
obtaining a base; and
adding an effective amount of the base to the colloidal suspension of meloxicam to change the pH of the colloidal suspension of meloxicam to at least 8.

Aspect 40. The method of Aspect 39, further comprising:
mixing the medicated stock colloidal suspension with water.

Aspect 41. A method for treating illness in an animal, comprising:
forming a medicated water-based colloidal suspension,
   wherein the forming the medicated water-based colloidal suspension comprises:
      the method of solubilizing meloxicam in a stock colloidal suspension according to Aspect 40; and
providing the medicated water-based colloidal suspension to the animal.

Aspect 42. The method of Aspect 41, wherein the animal is at least one of a pig, poultry, a cattle, a sheep, a horse, a dog, or a cat.

Aspect 43. A method for solubilizing piroxicam in water, comprising:
obtaining the piroxicam;
obtaining the water;
obtaining a hydrotrope; and
forming a colloidal suspension of piroxicam,
   wherein the forming of the colloidal suspension of piroxicam comprises:
      mixing the piroxicam and the hydrotrope in the water; and
      forming an aqueous colloidal suspension of piroxicam from the colloidal suspension of meloxicam,
         wherein the forming of the aqueous colloidal suspension of piroxicam comprises:
         obtaining a base; and
         adding an effective amount of the base to the colloidal suspension of piroxicam to change the pH of the colloidal suspension of piroxicam to be greater than 7.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

1. A method for solubilizing meloxicam in water, comprising:
obtaining the meloxicam;
obtaining the water;
obtaining a hydrotrope; and
forming a colloidal suspension of meloxicam,
wherein the forming of the colloidal suspension of meloxicam comprises:
mixing the meloxicam and the hydrotrope in the water; and
forming an aqueous colloidal suspension of meloxicam from the colloidal suspension of meloxicam,
wherein the forming of the aqueous colloidal suspension of meloxicam comprises:
obtaining a base; and
adding an effective amount of the base to the colloidal suspension of meloxicam to change the pH of the colloidal suspension of meloxicam to be greater than 7.

2. The method of claim 1, wherein the aqueous colloidal suspension of meloxicam includes:
the meloxicam present at least in an amount of 7.15×10⁻⁶ g per ml of the aqueous colloidal suspension of meloxicam.

3. The method of claim 1, wherein the aqueous colloidal suspension of meloxicam includes:
the hydrotrope being present in an amount of 0.001 g to 0.6 g per ml of the aqueous colloidal suspension of meloxicam.

4. The method of claim 1, wherein the aqueous colloidal suspension of meloxicam has a meloxicam concentration of 0.000715% to 12%, wherein the meloxicam concentration is based on the formula, 100× (mass of meloxicam/volume of the aqueous colloidal suspension of meloxicam).

5. The method of claim 1, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

6. The method of claim 1, wherein the hydrotrope comprises an alkali metal salt.

7. The method of claim 1, wherein the hydrotrope comprises at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N,N-dimethyl benzamide, paraaminobenzoic acid hydrochloride, procaine hydrochloride, sodium alkanoate, urea, N,N-dimethyl urea, resorcinol, pyrogallol, catechol, a,b-napthols,N-diethylnicotinamide (DENA), N,N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

8. The method of claim 1, wherein the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

9. The method of claim 1, wherein the forming of the aqueous colloidal suspension of meloxicam further comprises:
obtaining a pH buffer; and
adding the pH buffer to the colloidal suspension of meloxicam.

10. The method of claim 9, wherein the aqueous colloidal suspension of meloxicam includes:
the pH buffer present in an amount of 0.001 g to 0.14 g per mL of the aqueous colloidal suspension of meloxicam.

11. The method of claim 10, wherein the aqueous colloidal suspension of meloxicam includes:
the meloxicam present at least in an amount of 7.15×10⁻⁶ g per ml of the aqueous colloidal suspension of meloxicam,
the hydrotrope being present in an amount of 0.001 g to 0.6 g per mL of the aqueous colloidal suspension of meloxicam; and
the pH buffer present in an amount of 0.001 g to 0.14 g per mL of the aqueous colloidal suspension of meloxicam.

12. The method of claim 9, wherein the pH buffer comprises at least one of glycine, arginine, or a combination thereof.

13. The method of claim 9, wherein the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

14. The method of claim 1, wherein the obtaining the meloxicam comprises obtaining a tablet comprising the meloxicam as an active pharmaceutical ingredient (API).

15. A method for treating illness in an animal, comprising:
forming a medicated water-based colloidal suspension,
wherein the forming the medicated water-based colloidal suspension comprises:
the method of solubilizing meloxicam in water according to claim 1; and
providing the medicated water-based colloidal suspension to the animal.

16. An aqueous colloidal suspension of meloxicam, comprising:
water;
meloxicam,
wherein the meloxicam is present at least in an amount of 7.15×10⁻⁶ g per mL of the water;
a hydrotrope,
wherein the hydrotrope is present in an amount of 0.001 g to 0.6 g per mL of the water; and
a base,
wherein the aqueous colloidal suspension of meloxicam has a pH of greater than 7.

17. The aqueous colloidal suspension of meloxicam of claim 16, comprising a concentration of the meloxicam of 0.000715% to 12%.

18. The aqueous colloidal suspension of meloxicam of claim 16, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

19. A method for solubilizing meloxicam in a stock colloidal suspension, comprising:
obtaining the meloxicam;
obtaining the stock colloidal suspension;
obtaining a hydrotrope;
forming a colloidal suspension of meloxicam,
wherein the forming of the colloidal suspension of meloxicam comprises:
mixing the meloxicam and the hydrotrope in the stock colloidal suspension; and
forming a medicated stock colloidal suspension from the colloidal suspension of meloxicam,
wherein the forming of the medicated stock colloidal suspension comprises:
obtaining a base; and
adding an effective amount of the base to the colloidal suspension of meloxicam to change the pH of the colloidal suspension of meloxicam to at least 8.

20. A method for solubilizing piroxicam in water, comprising:
obtaining the piroxicam;
obtaining the water;
obtaining a hydrotrope; and
forming a colloidal suspension of piroxicam,
wherein the forming of the colloidal suspension of piroxicam comprises:
mixing the piroxicam and the hydrotrope in the water; and
forming an aqueous colloidal suspension of piroxicam from the colloidal suspension of meloxicam,
wherein the forming of the aqueous colloidal suspension of piroxicam comprises:
obtaining a base; and
adding an effective amount of the base to the colloidal suspension of piroxicam to change the pH of the colloidal suspension of piroxicam to be greater than 7.
